(19) United States
(12) Patent Application Publication
(10) Pub. No.: US 2004/0019316 A1
(43) Pub. Date: Jan. 29, 2004

(54) Nasal airway delivery, decongestion system (NADDS)

(76) Inventors: John K. Morris, Ann Arbor, MI (US); Lawrence Marentette, Ann Arbor, MI (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/387,624

(22) Filed: Mar. 13, 2003

Related U.S. Application Data
(60) Provisional application No. 60/363,921, filed on Mar. 13, 2002.

(51) Int. Cl. A61F 13/20

(52) U.S. Cl. 604/11

(57) ABSTRACT

This invention improves upon existing solutions by providing a nasal airway delivery decongestion apparatus in the form of a paddle-shaped tampon supported on an elongated handle, and a topical steroid disposed on the surface of the tampon, enabling the article to be inserted into the nasal passageway adjacent a local inferior turbinate mucosa for a sufficient period of time to facilitate extended relief. In the preferred embodiment, the tampon is sponge-like, enabling it to be impregnated with a topical steroid and solution, then dried and compressed into the paddle-like shape. Following insertion, the sponge expands, releasing the steroid to the local inferior turbinate mucosa. Although other topical steroids may be used, in the preferred embodiment Nasonex, or its equivalent, is the desired choice. In a further alternative embodiment, two paddle-shape tampons of the type disclosed and described herein are enjoined on a U-shaped handle enabling simultaneous insertion and administration of the topical steroid through both nostrils.

### REFERENCE TO RELATED APPLICATION

This application claims priority from U.S. Provisional Patent Application Serial No. 60/363,921, filed Mar. 13, 2002, the entire content of which is incorporated herein by reference.

### FIELD OF THE INVENTION

This invention relates generally to decongestant applicators and, in particular, to an insertion device impregnated with a topical steroid, enabling the steroid to be applied directly to the inferior turbinates.

### BACKGROUND OF THE INVENTION

Nasal congestion is endemic in our population. Allergic rhinitis causes a reactive swelling of the nasal mucosa, particularly the inferior turbinate, which impedes the normal flow of air through the nasal cavity. This, in turn causes difficulty in nasal breathing (preferred) vs. mouth breathing (not preferred). Patients with this condition have particular difficulty sleeping in the preferred recumbent position as venous congestion becomes more pronounced when recumbent. The nasal passages become relatively more dependent compared to the upright position. Mouth breathing may become a necessity. This may result in snoring, sleep apnea, poor humidification of inspired air and other upper airway disturbances.

Various remedies have proliferated including systemic decongestants (Phenylpropanolamine, Pseudoephedrine), vasoconstricting nasal sprays, steroid nasal sprays, nasal surgeries (septoplasty, turbinate cauterization, partial turbinate resection), “sinus” pillows, sleeping in the upright position in a reclining chair, etc. Recently Phenylpropanolamine (PPA) has been withdrawn from the market because of a positive association with stroke. Of concern is that Pseudoephedrine causes nasal decongestion through vasoconstriction, the same mechanism of action as PPA. The vasoconstricting nasal sprays have a long and poor track record of causing a condition, rhinitis medicamentosa, in which the mucosa responds with transient reduction in size, allowing easier breathing, followed by a hyperemic rebound phase that results in progressively more congestion and distress.

Steroid nasal sprays are currently recommended by most specialists and are moderately effective in many cases, often used in combination with oral antihistamines. Longer chain steroids that limit systemic absorption have been helpful in limiting systemic complications. Nasal spray patients tend to have the desired mucosal shrinkage anteriorly but persistent swelling posteriorly where delivery of the steroid is lacking. Nasal surgery also has been moderately successful but many patients are plagued with recurrent mucosal swelling and recongestion of the nasal airway years after an initial period of relative success. Some patients may not be a candidate for repeat turbinate cauterization. Sleeping in the upright position is sub optimal from many respects. Thus there is still a need for a more effective nasal congestion treatment.

### SUMMARY OF THE INVENTION

This invention improves upon existing solutions by providing a nasal airway delivery decongestion apparatus in the form of a paddle-shaped tampon supported on an elongated handle, and a topical steroid disposed on the surface of the tampon, enabling the article to be inserted into the nasal passageway adjacent a local inferior turbinate mucosa for a sufficient period of time to facilitate extended relief.

In the preferred embodiment, the tampon is sponge-like, enabling it to be impregnated with a topical steroid and solution, then dried and compressed into the paddle-like shape. Following insertion, the sponge expands, releasing the steroid to the local inferior turbinate mucosa.

Although other topical steroids may be used, in the preferred embodiment Nasonex, or its equivalent, is the desired choice. In a further alternative embodiment, two paddle-shape tampons of the type disclosed and described herein are enjoined on a U-shaped handle enabling simultaneous insertion and administration of the topical steroid through both nostrils.

### BRIEF DESCRIPTION OF DRAWINGS

FIG. 1A illustrates the first portion of an insertion process according to the invention;

FIG. 1B illustrates a second step in the insertion process;

FIG. 1C shows the article of the invention properly positioned relative to the inferior turbinate mucosa;

FIG. 2 is a coronal section of the head with the NADDS tampon in place;

FIG. 3A is an end view of an alternative embodiment of the invention showing how two tampons may be joined for simultaneous administration into both nostrils in a pre-insertion compressed/dried condition;

FIG. 3B is the end view of the device of FIG. 3A in a post-insertion state, wherein the sponge has been expanded to release the steroids to local inferior turbinate mucosa;

FIG. 3C is an end view of the device of FIG. 3A;

FIG. 3D is an end view of the device of FIG. 3B;

FIG. 3E is a top view of the device of FIGS. 3A and 3C; and

FIG. 3F is a top view of the device of FIGS. 3B and 3D.

### DETAILED DESCRIPTION OF THE INVENTION

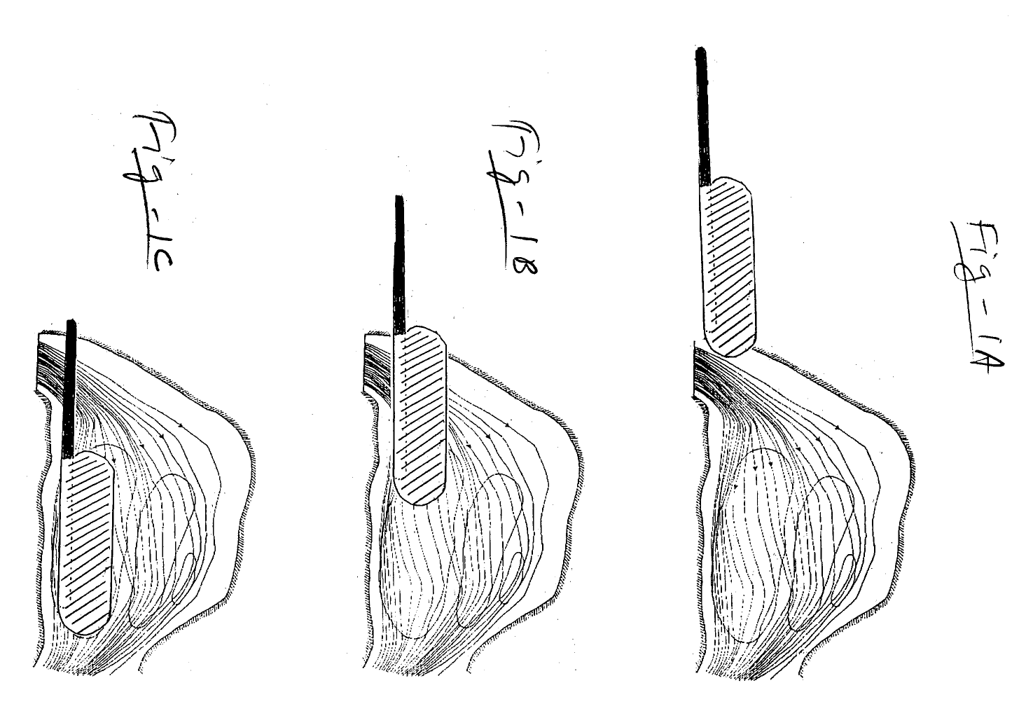

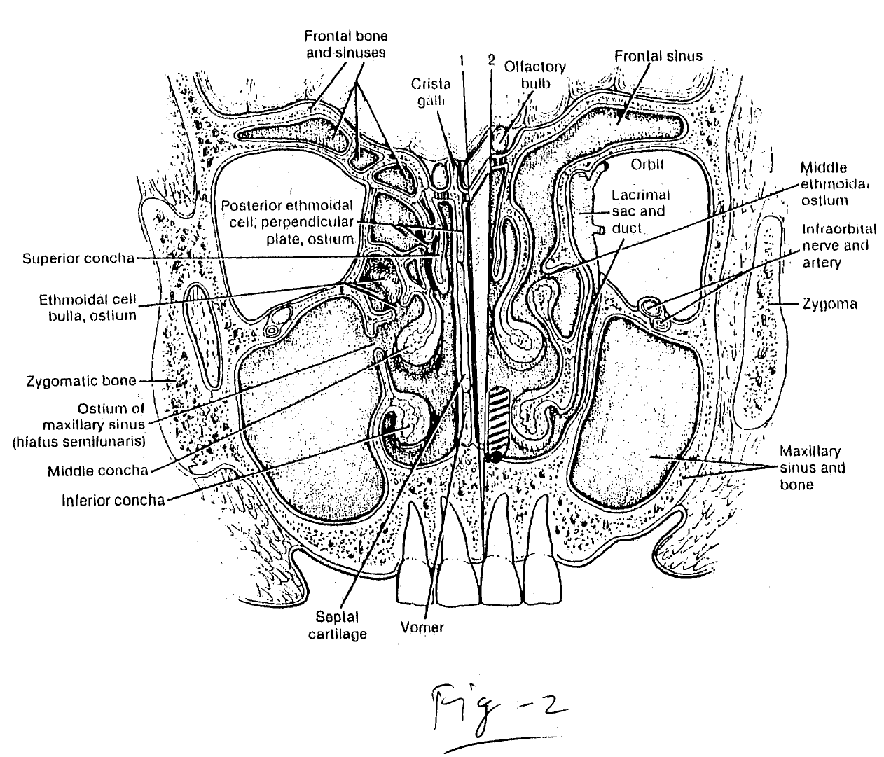

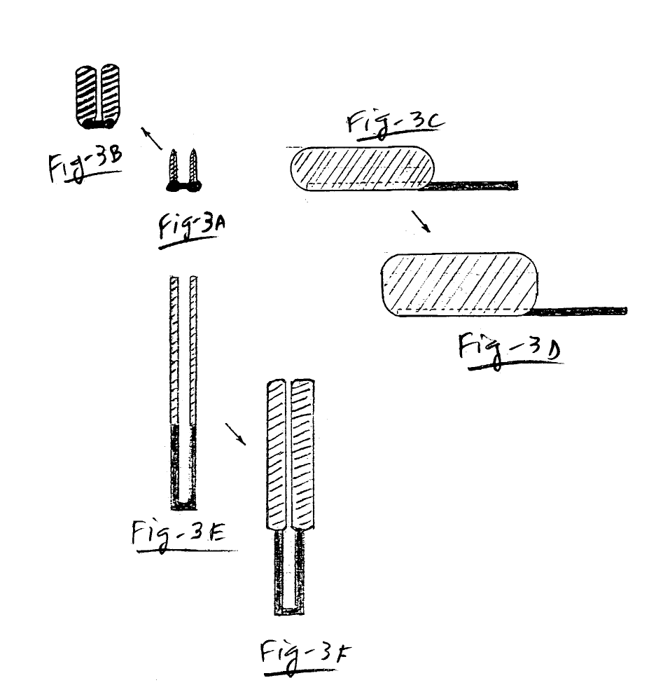

The current prototype uses a nasal tampon (NADDS) in each nostril, placed along the inferior turbinates for about 10 minutes before reclining for sleep. The insertion process is shown in the three progressive drawings of FIG. 1. FIG. 2 is a coronal section of the head with the NADDS tampon in place. FIG. 3 shows how two of the tampons may be joined for simultaneous administration of both nostrils.

The NADDS is impregnated with a topical steroid, preferably 0.1 mg Nasonex. This has consistently resulted in a decongestion of the nasal airway, allowing a full night's sleep with minimal nasal airway obstruction. In the preferred embodiment, a topical, long-chain steroid would be used on a tampon as a delivery vehicle to the most obstructed portion of the inferior nasal airway. This should lead to local mucosal shrinkage secondary to the topical steroid effect. Although over 50 percent of airflow occurs above the inferior turbinate, 50-60 percent of flow at the lower half of the posterior choana can be obstructed by the inferior turbinate.

The NADDS would be broad, flat and easily inserted by the user. The material of the pad portion is preferably sponge-like, enabling it to be impregnated with the topical steroid in solution, then dried and compressed into a flat paddle-like shape. Following insertion, the sponge expands, releasing the steroid to local inferior turbinate mucosa.

Video endoscopic techniques and nasal flow measuring devices exist to document pre- and post-device deployment flow rates/resistances. This could result in a marketable NADDS product that would be prescribed by ENT and Allergy specialists for use in the nasally congested population. A pharmaceutical company would the logical manufacturer/distributor. Nasal measurements may be necessary to prescribe the correct size(s). Patient education would be necessary to provide for a safe application of the device. Ongoing monitoring would likely be necessary to be vigilant for possible complications of treatment. A moderately affected patient may use a pair of NADDS daily in conjunction with a nasal spray.

We claim:

1. Nasal airway delivery and decongestion apparatus, comprising:
a paddle-shaped tampon supported on an elongated handle, the dimensions of the tampon and handle being such that when inserted into a human nasal passageway, the tampon is adjacent to local inferior turbinate mucosa; and
a topical steroid disposed on the tampon.

2. The apparatus of claim 1, wherein the tampon is sponge-like, enabling it to be impregnated with the topical steroid in solution, then dried and compressed into the flat paddle-like shape, such that following insertion, the sponge expands, releasing the steroid to the local inferior turbinate mucosa.

3. The apparatus of claim 1, wherein the topical steroid is Nasonex or the equivalent.

4. The apparatus of claim 1, further including two of the tampons joined on a U-shaped handle enabling simultaneous administration through both nostrils.
